(19) United States
(12) Patent Application Publication
(10) Pub. No.: US 2012/0093947 A1
(43) Pub. Date: Apr. 19, 2012

(54) METHOD OF TREATING REACTIVE AIRWAY DISEASE

(75) Inventors: Bradley E. Britigan, Cincinnati, OH (US); Krzysztof J. Reszka, Cincinnati, OH (US); Dennis W. McGraw, Cincinnati, OH (US)

(73) Assignees: UNITED STATES DEPARTMENT OF VETERANS AFFAIRS, Washington, DC (US); UNIVERSITY OF CINCINNATI, Cincinnati, OH (US)

(21) Appl. No.: 13/203,500

(22) PCT Filed: Mar. 1, 2010
(86) PCT No.: PCT/US2010/025730

§ 371 Date: Oct. 5, 2011

Related U.S. Application Data
(60) Provisional application No. 61/156,101, filed on Feb. 27, 2009.

(51) Int. Cl. A61K 33/04; A61K 31/167; A61P 11/00; A61K 31/4164; A61P 11/06; A61K 31/137; A61K 31/4704

(52) U.S. Cl. 424/713; 514/653; 514/651; 514/629; 514/312; 514/392

(57) ABSTRACT

A method of treating a reactive airway disease in a subject comprising administering at least one peroxidase inhibitor in association with administration of at least one β-agonist.

The present invention relates generally to treatment of reactive airway disease, particularly with β₂-agonists and peroxidase inhibitors.

Asthma is an increasing health problem among both children and adults. Asthma is a chronic inflammatory disease characterized by bronchial smooth muscle contraction and episodic narrowing of the airway.

Elevated levels of inflammatory cells, particularly neutrophils (PMN) and eosinophils (EOS) and their secretory products, are present in asthmatic airways and increase during clinical exacerbations of the disease. EOS and PMN generate large amounts of superoxide (O₂^{•-}) and hydrogen peroxide (H₂O₂) and release their granule contents, which include the unique peroxidases eosinophil peroxidase (EPO) (by EOS) and myeloperoxidase (MPO) (by PMN). Peroxidases, like MPO, EPO, and the endogenous airway peroxidase, LPO (lactoperoxidase), commonly utilize endogenously-generated H₂O₂ to convert substrates such as tyrosine (TyrOH), SCN⁻, NO₂, Br⁻ and Cl⁻ to reactive metabolites that interact with cell components, causing their modification and resulting in loss of normal physiological functions.

β₂-agonists are commonly used to treat asthma. Given that β₂-agonists, including salbutamol, fenoterol, and terbutaline, also possess the phenolic character of tyrosine and function in the oxidizing environment of inflamed airways, they too can be metabolized by airway peroxidases. Peroxidase-mediated oxidation causes structural modification of the β₂-agonists and makes them less active and less effective. Consequently, therapeutic activity and effectiveness of β₂-agonists might decrease during times of increased airway inflammation that characterize acute asthma exacerbations.

In asthmatic airways, there is also increased synthesis of NO^{•}. Inflamed airways are a source of NO₂⁻, which is an excellent substrate for peroxidases. In the presence of NO₂⁻, airway peroxidases convert salbutamol and other β₂-agonists to nitrated products with reduced affinity to bind to the β₂-AR receptor and impaired ability to generate cAMP when compared to the β₂-agonists.

β₂-agonists may fail to relieve bronchospasm and may be less effective in relieving bronchoconstriction when asthma exacerbations are severe (status asthmaticus). Oxidation and nitration of salbutamol and other β₂-agonists by peroxidases, hydrogen peroxide, and NO₂⁻ present in inflamed airways may be the mechanisms that underlie impaired β₂-agonist efficacy.

There is a need to develop an improved method of treating asthma and other reactive airway diseases that will enhance the ability of β₂-agonists to relieve bronchospasm, improve bronchodilation, and decrease the risk of death.

Accordingly, the present invention provides a method of treating a reactive airway disease.

One embodiment of the invention provides a method of treating a reactive airway disease in a subject comprising administering at least one peroxidase inhibitor in association with administration of at least one β₂-agonist.

Another embodiment of the invention provides a pharmaceutical composition comprising at least one β₂-agonist, at least one peroxidase inhibitor in an amount effective to inhibit metabolism of the at least one β₂-agonist, and a pharmaceutically acceptable carrier.

Another embodiment of the invention provides an inhaler comprising at least one β₂-agonist and at least one peroxidase inhibitor in an amount effective to inhibit metabolism of the at least one β₂-agonist.

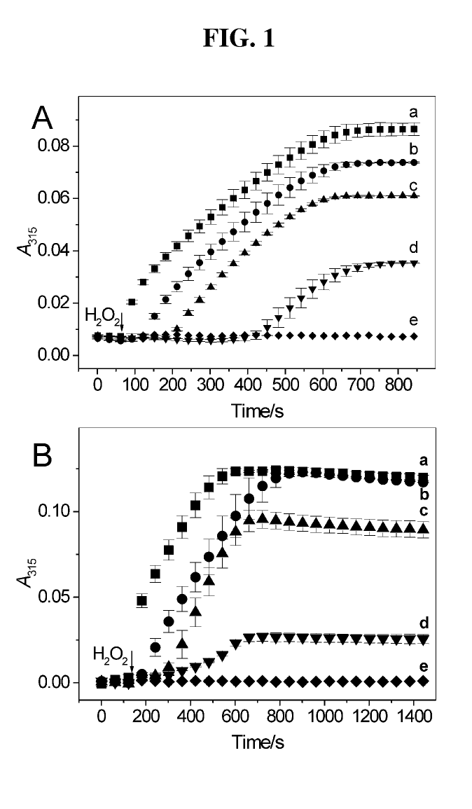

FIG. 1 is a graphical representation of the effect of ascorbic acid on oxidation of salbutamol (section A) and fenoterol (section B) by MPO/H₂O₂.

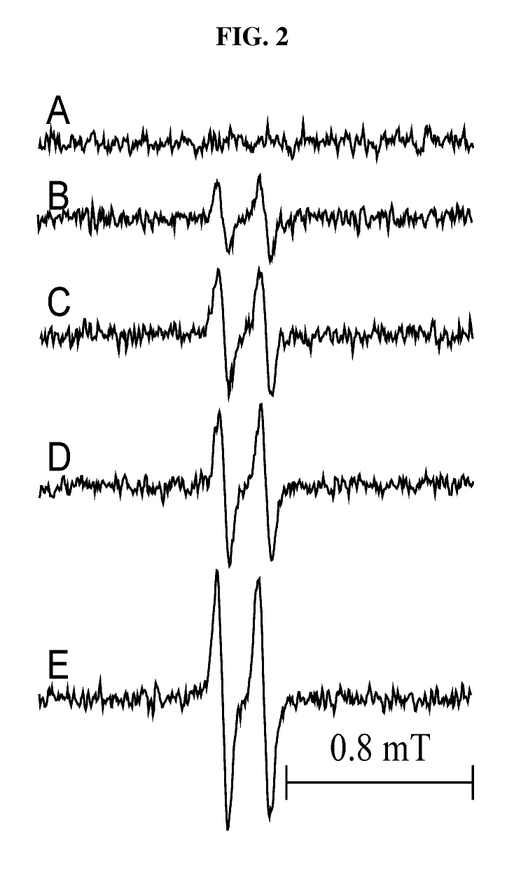

FIG. 2 is a graphical representation of the formulation of ascorbate radicals in pH during oxidation of β₂-agonists by MPO/H₂O₂.

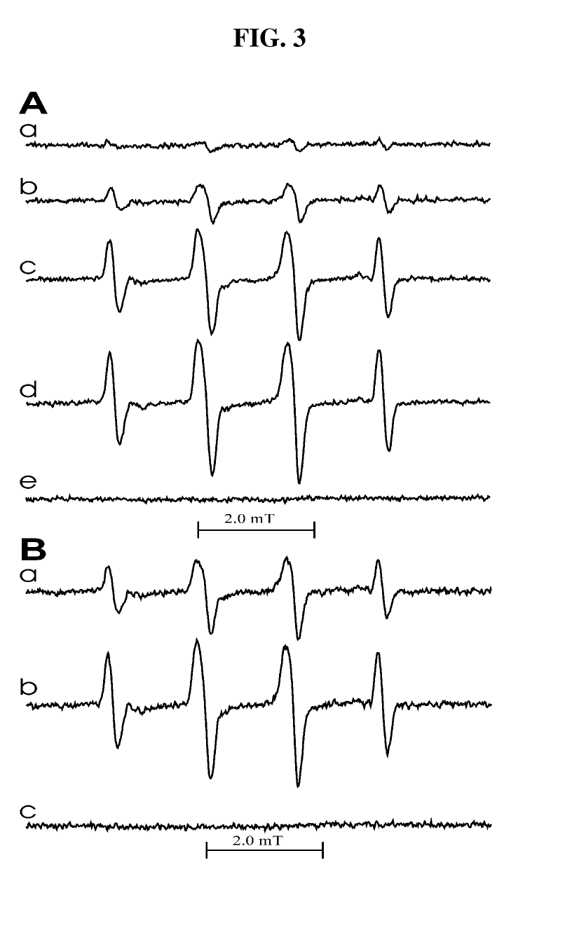

FIG. 3 is a graphical representation of the effect of glutathione on oxidation of of salbutamol (section A) and fenoterol (section B) by MPO/H₂O₂.

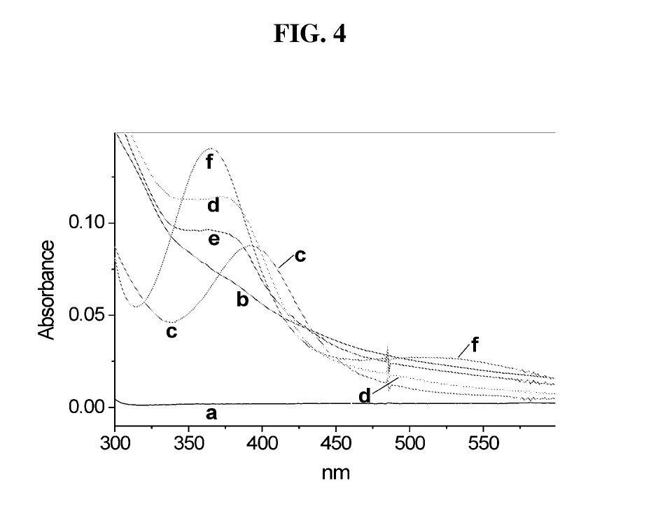

FIG. 4 is a graphical representation of the effect of GSH and BSA on the oxidation of fenoterol and terbutaline by MPO/H₂O₂.

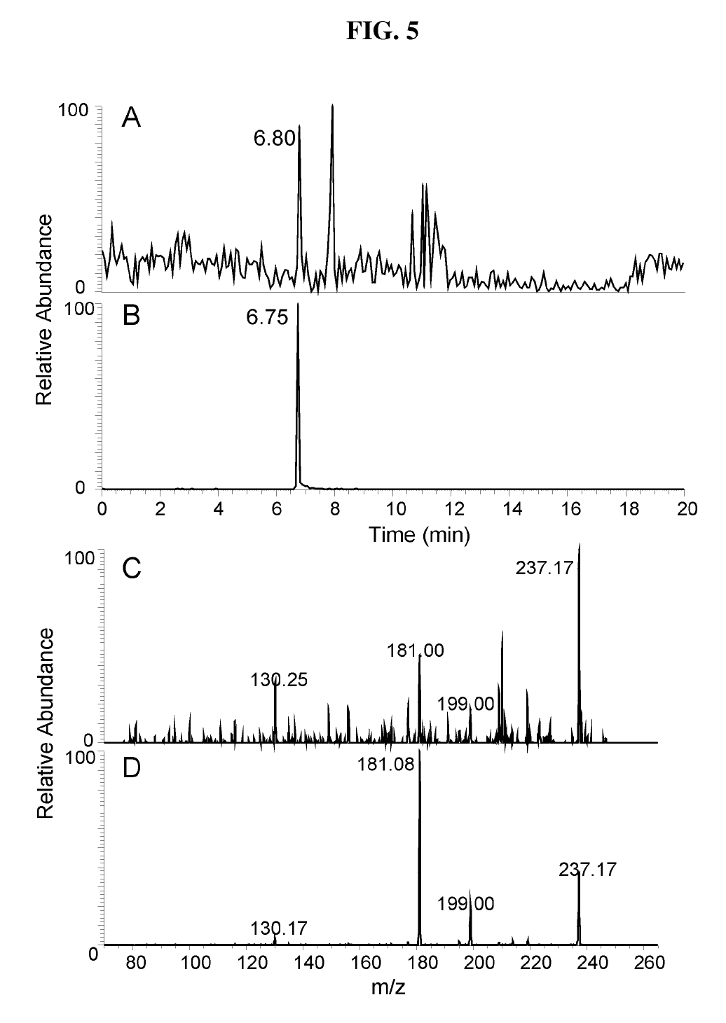

FIG. 5 is a graphical representation of HPLC elution profile of molecular ions of m/z 255.13 in breath condensate of an asthma patients treated with salbutamol (trace A, retention time 6.80 min) and from a salbutamol nitrated in vitro (with MPO(LPO)/H₂O₂/NO₂⁻) (trace B, retention time 6.75 min) and MS/MS spectra of the parent ion of m/z 255.13 in breath condensate (trace C) and from salbutamol nitrated in vitro (trace D).

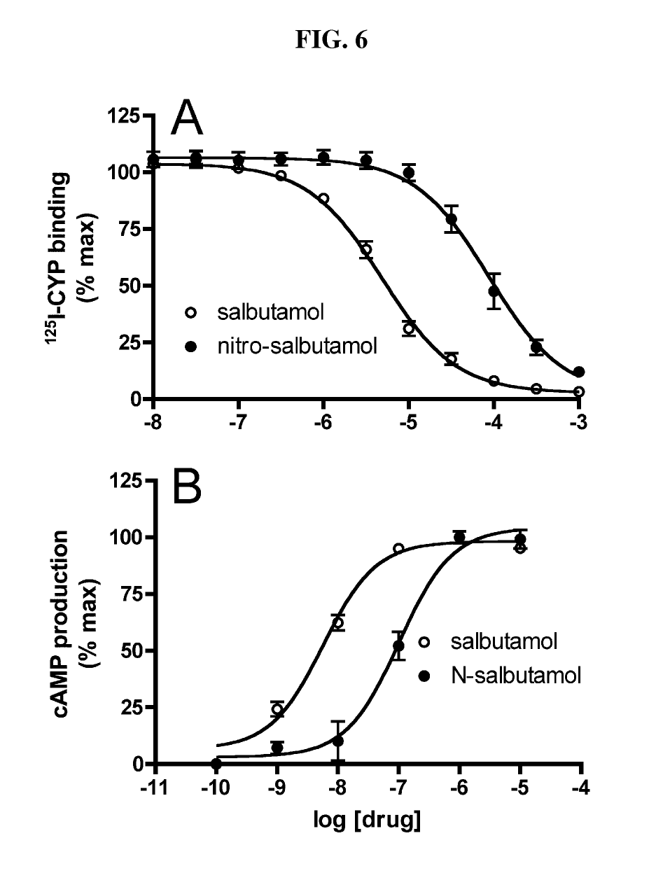

FIG. 6 is a graphical representation of ¹²⁵I-CYP displacement from β₂-receptors by intact (∘) and nitrated () salbutamol (section A) and cAMP production by smooth muscle cells stimulated by intact (∘) and nitrated () salbutamol (section B).

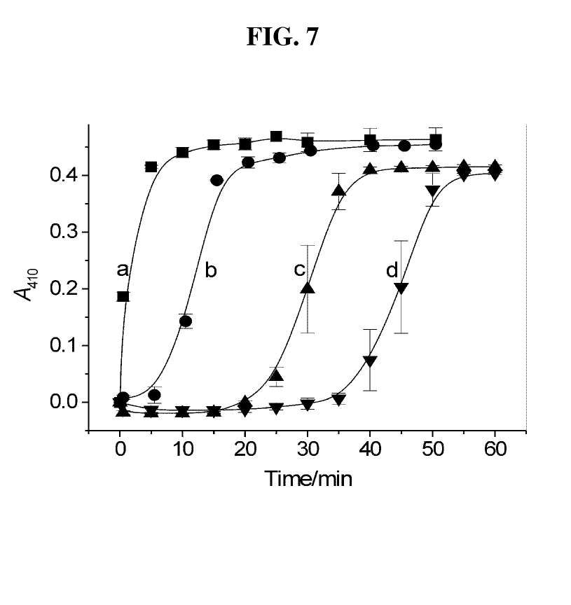

FIG. 7 is a graphical representation of the effect of ascorbate on nitration of salbutamol by LPO/H₂O₂/NO₂⁻.

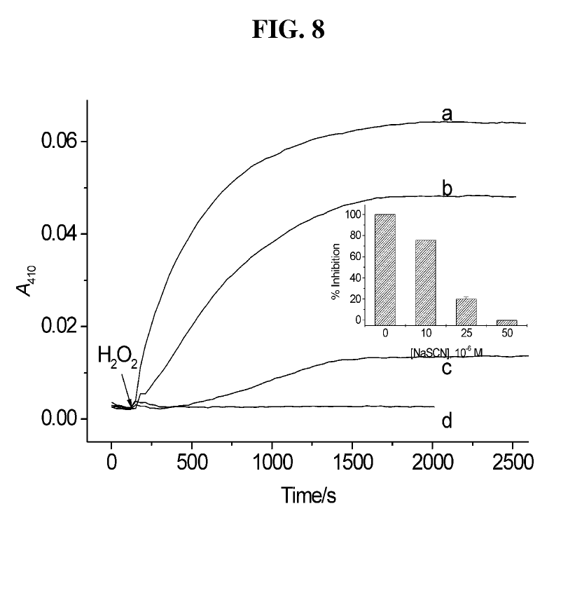

FIG. 8 is a graphical representation of the effect of NaSCN on nitration of salbutamol by LPO/H₂O₂/NO₂⁻.

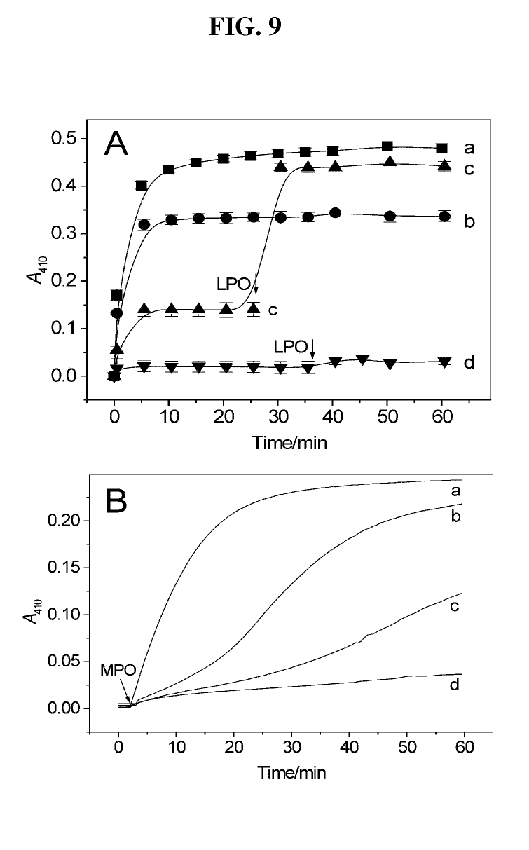

FIG. 9 is a graphical representation of the effect of methimazole on nitration of salbutamol by LPO/H₂O₂/NO₂⁻ (section A) and MPO/H₂O₂/NO₂⁻ (section B) systems.

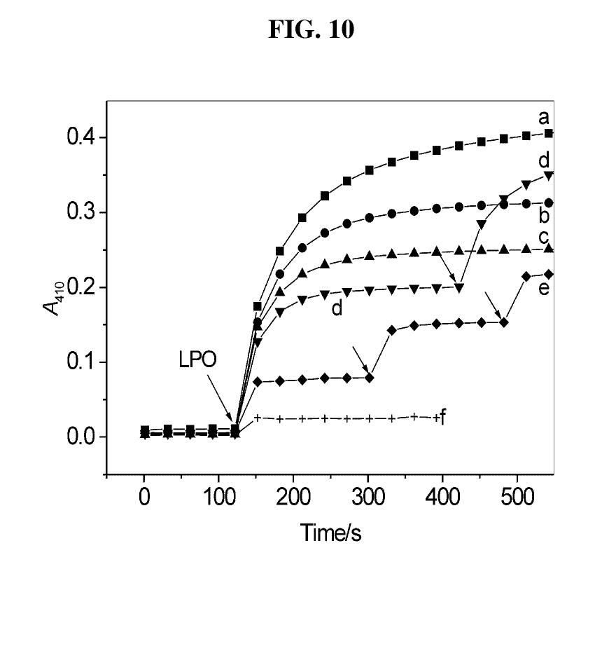

FIG. 10 is a graphical representation of the effect of dapsone on nitration of salbutamol by LPO/H₂O₂/NO₂.

“Reactive airway disease” means any acute or chronic disorder characterized by widespread and largely reversible reduction in the caliber of bronchi and bronchioles, due in varying degrees to smooth muscle spasm, mucosal edema, inflammation, and excessive mucus in the lumens of airways of the lung. Common symptoms of reactive airway disease are dyspnea, wheezing, and cough. In general, this term refers to the various forms of asthma, as well as acute or chronic bronchitis and emphysema.

“β₂-agonist” means any member of a group of drugs that bind to the β₂-adrenergic receptor of cells, which in turn activates adenylate cyclase and elevates cellular levels of cAMP. When β₂-agonists bind to smooth muscle cells, smooth muscle relaxation occurs, making β₂-agonists useful in the treatment of asthma and other forms of reactive airway disease. For this use, β₂-agonists are usually administered to lung via aerosol or nebulizer.

“Peroxidase” means any member of a family of enzymes that typically catalyze a reaction of the form ROOR′+electron donor (2 e⁻)+2H⁺→ROH+R′OH. Although the optimal substrate for peroxidases is often hydrogen peroxide, some of them use, and in some cases prefer, organic hydroperoxides, such as lipid peroxides. Peroxidases can contain a heme cofactor in their active sites, or redox-active cysteine or selenocysteine residues. Examples of peroxidases include myeloperoxidase, eosinophil peroxidase, thyroid peroxidase, lactoperoxidase, and horseradish peroxidase.

“Peroxidase inhibitor” means any compound that inhibits the activity of one or more peroxidases, as defined above. Inhibition can occur by either by inhibiting the enzyme itself or reacting with one of its products.

“Antioxidant” means any compound or enzyme that prevents or inhibits the oxidation of another molecule.

“Aerosol” means a suspension of solid or liquid particles in a gaseous medium. For the purposes of this invention, a compound that has been suspended in a gaseous medium has been “aerosolized.”

“Inhalation” means movement of air from the external environment, through the airway, and into the alveoli of the lungs. Inhalation can occur through the nose or mouth.

“Inhaler” means a device used for inhalation of medicine in the form of a vapor or gas. The term inhaler includes, but is not limited to, dry powder inhalers and metered dose inhalers.

“Nebulizer” means a device used for inhalation of a medicine the form of an aerosol or mist. A compound that has been prepared for administration via nebulizer has been “nebulized.”

Standard treatment of acute asthma exacerbations includes inhalation of β₂-agonists, which activate β₂-adrenergic receptors (hereinafter “β₂-AR”) on bronchial smooth muscle cells, triggering an increase in cyclic AMP that leads to smooth muscle relaxation. β₂-agonists are the most potent bronchodilators available and are used to relieve acute airway bronchospasm. Known β₂-agonists include both short-acting β₂-agonists (e.g., salbutamol, fenoterol, isoproterenol, and terbutaline) and long-acting β₂-agonists (e.g., salmeterol, formoterol, arformoterol, and indacaterol). However, β₂-agonists sometimes fail to relieve bronchospasm and β₂-agonists also appear to be much less effective in relieving bronchoconstriction when asthma exacerbations are severe (status asthmaticus), which may increase the risk of death. See. e.g., Spitzer et al. (1992) N. Engl. J. Med. 326:501-506; Suissa et al. (1994) Am. J. Respir. Crit. Care Med. 149(3):604-610; Abramson (2003) Am. J. Respir. Med. 2(4):287-297.

Inflammation is an important component of asthma. During asthma exacerbations, EOS and PMN generate large amounts of superoxide (O₂^{•-}) and hydrogen peroxide (H₂O₂) and release eosinophil peroxidase (EPO) and myeloperoxidase (MPO), heme enzymes that functionally resemble the lactoperoxidase (LPO) that is normally present in lung lining fluid and plays a protective role against pathogens. MPO, EPO, and LPO utilize endogenously-generated H₂O₂ to convert substrates such as tyrosine (TyrOH), SCN⁻, NO₂⁻, Br⁻ and Cl⁻ to reactive metabolites that interact with cell components, causing their modification and resulting in loss of normal physiological functions. Treatment of PMN and EOS with β₂-agonists such as salbutamol and fenoterol inhibits superoxide production and degranulation. See, e.g., Yasui et al. (2006) Int. Arch. Allergy Immunol. 139(1):1-8; Tachibana et al. (2002) Clin. Exp. Immunol. 130(3):415-423.

All commercially available β₂-agonists are phenols or polyphenols. Phenols are peroxidase substrates and oxidation of the phenolic moiety can produce phenoxyl radicals, which dimerize or react with other cellular targets leading to oxidative injuries to lungs. Oxidation of the phenolic moiety can be described by reactions given by equations 1-3, below, with MPO as a representative peroxidase and TyrOH as a substrate. The immediate metabolite of TyrOH is the tyrosyl radical (TyrO^{•}).

MPO+H₂O₂→MPO—I+H₂O (1)

MPO—I+TyrOH→MPO-II+Tyr^{•} (2)

MPO-II+TyrOH→MPO+Tyr^{•} (3)

Tyr^{•}+Tyr^{•}→→(TyrOH)₂ dimer (4)

In the absence of other substrates, phenoxyl radicals typically form dimers through o,o′-biphenyl or p-phenoxyphenyl ether linkages (e.g., equation 4). See, e.g., Sawahata et al. (1982) Biochem. Biophys. Res. Communs. 109(3):988-994; Yu et al. (1994) Environ. Sci. Technol. 28:2154-2160. The Tyr^{•} radicals also react with other targets such as tyrosine residues in proteins or react with reduced glutathione causing its oxidation. See, e.g., Heinecke et al. (1993) J. Clin. Invest. 91:2866-2872; Tien (1999) Arch. Biochem. Biophys. 367(1):61-66. Oxidation of phenolics by LPO and EPO systems occurs, in principle, according to the same mechanism as that associated with MPO.

Given that β₂-agonists, including salbutamol, fenoterol, and terbutaline, possess the phenolic character of tyrosine and function in the oxidizing environment of inflamed airways, they too can be metabolized by airway peroxidases. Such peroxidase-mediated oxidation causes structural modification of the drugs similar to that reported for tyrosine. Because the therapeutic activity of β₂-agonists is dependent upon β₂-agonist affinity for and ability to bind to β₂-AR, traits which are dependent on β₂-agonist structure, structural modification of β₂-agonists impacts the therapeutic activity of β₂-agonists, making them less active and less effective during the times of increased airway inflammation that characterize acute asthma exacerbations. Oxidation of β₂-agonists is totally dependent on simultaneous presence of peroxidase and H₂O₂, indicating that oxidation of β₂-agonists requires an active peroxidase. Reszka et al. (2009) Chem. Res. Toxicol. 22(6):1137-1150.

The above suggested to us that inhibiting the peroxidases present in an airway may be therapeutic and may improve the efficacy of β₂-agonists in treating asthma and other reactive airway diseases.

Nitric oxide (NO^{•}) is a potent relaxant of smooth muscle. It is generated by constitutive and inducible forms of nitric oxide synthases (NOS), which are present in many cell types within the respiratory tract, including airway and alveolar epithelial cells, macrophages, neutrophils, mast cells, and vascular endothelial and smooth muscle cells. The concentration of NO^{•} in airways is normally in the range of 10-20 μM, but is elevated during inflammation. Overproduction of NO^{•} does not lead to airway relaxation, but instead may contribute to airway narrowing and disease severity. In asthmatic airways, there is increased synthesis of NO^{•}.

In addition to constituting an oxidizing environment, inflamed airways also constitute a nitrating environment. As noted above, salbutamol, because of its phenolic character, is a potential substrate for peroxidative metabolism. Inflamed airways are a source of NO₂⁻, an excellent substrate for peroxidases. Oxidation of NO₂⁻ can be described by a set of equations similar to those described earlier.

MPO—I+NO₂⁻→MPO—II+NO₂^{•} (5)

MPO—II+NO₂⁻→MPO+NO₂^{•} (6)

In the presence of NO₂, airway peroxidases convert salbutamol to nitrated products with reduced affinity to bind to the β₂-AR receptor and impaired ability to generate cAMP when compared to the native drug. Utilization of salbutamol by peroxidases and NO₂⁻ present in an inflamed airway may be a mechanism that underlies impaired β₂-agonist efficacy in certain clinical settings.

The above suggested to us that inhibiting the nitration of β₂-agonists by NO₂⁻ may be therapeutic and may improve the efficacy of β₂-agonists in treating asthma and other reactive airway diseases.

Exemplary embodiments of a method for treating a reactive airway disease are hereinafter described.

One exemplary embodiment of the invention comprises a method of treating a reactive airway disease in a subject comprising administering at least one peroxidase inhibitor in association with administration of at least one β₂-agonist.

β₂-agonists can be metabolized by peroxidase and NO₂⁻. Such metabolism renders β₂-agonists less active and less effective in treating reactive airway disease. Administration of at least one peroxidase inhibitor in association with administration of at least one β₂-agonist can inhibit metabolism of β₂-agonists, thereby preserving the activity and effectiveness of such β₂-agonists. The amount of the at least one peroxidase inhibitor administered depends on the specific peroxidase inhibitor(s) administered, but should be such that the concentration of the peroxidase inhibitor(s) delivered to the airway lining fluid inhibits metabolism of the at least one β₂-agonist. For example, the concentration of dapsone necessary to inhibit metabolism of β₂-agonists in the airway lining fluid is in the range of approximately 50 μM to approximately 1 mM; the concentration of methimazole necessary to inhibit metabolism of β₂-agonists in the airway lining fluid is in the range of approximately 20 μM to approximately 200 μM; the concentration of thiocyanate necessary to inhibit metabolism of β₂-agonists in the airway lining fluid is in the range of approximately 10 μM to approximately 200 μM. Persons of ordinary skill in the art will be able to determine effective concentrations for other peroxidase inhibitors through routine experimentation.

In a specific exemplary embodiment of the method of treating a reactive airway disease, the reactive airway disease is selected from the group consisting of asthma, emphysema, and bronchitis.

In another specific exemplary embodiment of the method of treating a reactive airway disease, the β₂-agonist is selected from the group comprising salbutamol, fenoterol, terbutaline, isoproterenol, salmeterol, formoterol, arformoterol, and indacaterol.

In another specific exemplary embodiment of the method of treating a reactive airway disease, the peroxidase inhibitor is selected from the group consisting of methimazole, dapsone, and thiocyanate.

In another specific exemplary embodiment of the method of treating a reactive airway disease, administering the at least one peroxidase inhibitor comprises administering the at least one peroxidase inhibitor via inhalation.

β₂-agonists are typically administered via inhalation, either by inhaler or nebulizer. It is therefore preferred that administration of the at least one peroxidase inhibitor occur via inhalation. Such inhalation can occur via inhaler, including metered dose inhaler, and nebulizer. Inhalers and nebulizers are well known in the art, as are means of preparing aerosol formulations and solutions to be delivered by inhalers and nebulizers. Such formulations and solutions should be prepared so that, when administered, they deliver the at least one peroxidase inhibitor to the airway lining fluid in a concentration effective to inhibit metabolism of the at least one β₂-agonist.

In another specific exemplary embodiment of the method of treating a reactive airway disease, administering the at least one peroxidase inhibitor comprises administering the at least one peroxidase inhibitor via a metered dose inhaler or a nebulizer.

In another specific exemplary embodiment of the method of treating a reactive airway disease, administering at least one peroxidase inhibitor in association with administration of at least one β₂-agonist comprises administering the at least one peroxidase inhibitor concurrent with administration of the at least one β₂-agonist.

β₂-agonist are typically administered via inhalation, either by inhaler or nebulizer. Both the at least one β₂-agonist and the at least one peroxidase inhibitor may be administered concurrently from a single inhaler or a single nebulizer.

In another specific exemplary embodiment of the method of treating a reactive airway disease, administering at least one peroxidase inhibitor in association with administration of at least one β₂-agonist comprises administering the at least one peroxidase inhibitor separate from administration of the at least one β₂-agonist.

While β₂-agonists are typically administered via inhalation, peroxidase inhibitors may be administered via other routes. The at least one β₂-agonist and the at least one peroxidase inhibitor may be administered via separate inhalers. Persons of ordinary skill in the art will recognize that, while administration of both the at least one β₂-agonist and the at least one peroxidase inhibitor via inhalation is preferred, administration of either separately and via alternative routes is possible. For example, the at least one β₂-agonist may be administered via an inhaler and the at least one peroxidase inhibitor administered orally.

In another specific exemplary embodiment of the method of treating a reactive airway disease, administering at least one peroxidase inhibitor in association with administration of at least one β₂-agonist comprises administering the at least one peroxidase inhibitor prior to administration of the at least one β₂-agonist.

Again, though concurrent administration of the at least one β₂-agonist and the at least one peroxidase inhibitor is preferred, alternative administrations exist. In one example, the at least one peroxidase inhibitor is administered orally and systemically, thereby establishing a base level of peroxidase inhibitor in the airway lining fluid, while the at least one β₂-agonist is administered via inhalation on an as needed basis.

In another specific exemplary embodiment of the method of treating a reactive airway disease, the method further comprises administering at least one antioxidant in association with administration of at least one β₂-agonist.

β₂-agonists can be metabolized by peroxidase and NO₂⁻. Such metabolism renders β₂-agonists less active and less effective in treating reactive airway disease. Administration of at least one antioxidant in association with administration of at least one β₂-agonist can inhibit metabolism of β₂-agonists, thereby preserving the activity and effectiveness of such β₂-agonists. The amount of the at least one antioxidant administered depends on the specific antioxidant(s) administered, but should be such that the concentration of the antioxidant(s) delivered to the airway lining fluid inhibits metabolism of the at least one β₂-agonist. For example, the concentration of glutathione necessary to inhibit metabolism of β₂-agonists in the airway lining fluid is in the range of approximately 100 μM to approximately 1 mM; the concentration of ascorbate necessary to inhibit metabolism of β₂-agonists in the airway lining fluid is in the range of approximately 10 μM to approximately 1 mM. Persons of ordinary skill in the art will be able to determine effective concentrations for other antioxidants through routine experimentation.

In another specific exemplary embodiment of the method of treating a reactive airway disease, the method further comprises administering at least one antioxidant and the antioxidant is selected from the group consisting of ascorbate and glutathione.

In another specific exemplary embodiment of the method of treating a reactive airway disease, the method further comprises administering at least one antioxidant via inhalation.

β₂-agonists are typically administered via inhalation, either by inhaler or nebulizer. It is therefore preferred that administration of the at least one antioxidant occur via inhalation. Such inhalation can occur via inhaler, including metered dose inhaler, and nebulizer. Inhalers and nebulizers are well known in the art, as are means of preparing aerosol formulations and solutions for delivering such inhalers and nebulizers. Such formulations and solutions should be prepared so that, when administered, they deliver the at least one antioxidant to the airway lining fluid in a concentration effective to inhibit metabolism of the at least one β₂-agonist.

In another specific exemplary embodiment of the method of treating a reactive airway disease, the method further comprises administering at least one antioxidant via a metered dose inhaler or a nebulizer.

In another specific exemplary embodiment of the method of treating a reactive airway disease, the method further comprises administering at least one antioxidant concurrent with administration of the at least one β₂-agonist.

β₂-agonists are typically administered via inhalation, either by inhaler or nebulizer. Both the at least one β₂-agonist and the at least one peroxidase inhibitor may be administered concurrently from a single inhaler or a single nebulizer.

In another specific exemplary embodiment of the method of treating a reactive airway disease, the method further comprises administering at least one antioxidant separate from administration of the at least one β₂-agonist.

While β₂-agonists are typically administered via inhalation, antioxidants may be administered via other routes. The at least one β₂-agonist and the at least one antioxidant may be administered via separate inhalers. Persons of ordinary skill in the art will recognize that, while administration of both the at least one β₂-agonist and the at least one antioxidant via inhalation is preferred, administration of either separately via alternative routes is possible. For example, the at least one β₂-agonist may be administered via an inhaler and the at least one antioxidant administered orally.

In another specific exemplary embodiment of the method of treating a reactive airway disease, the method further comprises administering at least one antioxidant prior to administration of the at least one β₂-agonist.

Again, though concurrent administration of the at least one β₂-agonist and the at least one antioxidant is preferred, alternative administrations exist. In one example, the at least one antioxidant is administered orally and systemically, thereby establishing a base level of antioxidant in the airway lining fluid, while the at least one β₂-agonist is administered via inhalation on an as needed basis.

Another exemplary embodiment of the present invention comprises a pharmaceutical composition comprising at least one β₂-agonist, at least one peroxidase inhibitor in an amount effective to inhibit metabolism of the at least one β₂-agonist, and a pharmaceutically acceptable carrier.

In a specific exemplary embodiment of the pharmaceutical composition comprising at least one β₂-agonist, at least one peroxidase inhibitor in an amount effective to inhibit metabolism of the at least one β₂-agonist, and a pharmaceutically acceptable carrier, the composition is aerosolized or nebulized.

In another specific exemplary embodiment of the pharmaceutical composition comprising at least one β₂-agonist, at least one peroxidase inhibitor in an amount effective to inhibit metabolism of the at least one β₂-agonist, and a pharmaceutically acceptable carrier, the composition further comprises at least one antioxidant in an amount effective to inhibit metabolism of the at least one β₂-agonist.

In another specific exemplary embodiment of the pharmaceutical composition comprising at least one β₂-agonist, at least one peroxidase inhibitor in an amount effective to inhibit metabolism of the at least one β₂-agonist, and a pharmaceutically acceptable carrier, the composition further comprises at least one antioxidant in an amount effective to inhibit metabolism of the at least one β₂-agonist and the composition is aerosolized or nebulized.

Another exemplary embodiment of the present invention comprises an inhaler comprising at least one β₂-agonist and at least one peroxidase inhibitor in an amount effective to inhibit metabolism of the at least one β₂-agonist.

In a specific exemplary embodiment of the inhaler, the inhaler further comprises at least one antioxidant in an amount effective to inhibit metabolism of the at least one β₂-agonist.

The inventions herein should be considered in light of, but not limited by, the following examples.

### EXAMPLE 1

### Materials

Lactoperoxidase (LPO) from bovine milk (EC 1.11.1.7), catalase from bovine liver (EC 1.11.1.6; 2,350 U/mg), horseradish peroxidase (HRP), terbutaline hemisulfate, metaproterenol hemisulfate, L-tyrosine, and all other chemicals (hydrogen peroxide (30%), L-GSH, ascorbic acid, methimazole (1-methyl-3H-imidazole-2-thione), dapsone (diamino-diphenyl sulfone), L-methionine, NaSCN, NaCN, NaN₃, diethylenetriamine pentaacetic acid (DTPA), 2,2′-azino-di-(3-ethyl-benzthiazoline-6-sulphonic acid) (ABTS), 5,5-dimethyl pyrroline N-oxide (DMPO)), 2-methyl-2-nitrosopropane (MNP) and albumin (bovine serum, BSA)) are obtained from Sigma-Aldrich Co. (St. Louis, Mo.). LPO concentration is determined using ε₄₁₂ of 1.12×10⁵ M⁻¹ cm⁻¹. Jenzer et al. (1986) J. Biol. Chem. 261(33):15550-15556. Myeloperoxidase (MPO) from human leucocytes (lyophilized powder, 25 U, RZ (A₄₂₉/A₂₈₀) of 0.61) and SOD from bovine liver (5000 U/mg) are obtained from Axxora, LLC (San Diego, Calif.) and reconstituted with 0.25 mL of distilled water before use. Salbutamol hemisulfate, fenoterol hydrobromide, and glucose oxidase type X are from MP Biochemicals, Inc. (Solon, Ohio). α-D(+)-Glucose is from Across Organic (Belgium). Human EPO (460 U/mg protein) (Calbiochem) is reconstituted with 0.243 mL of water. All chemicals are used as received. H₂O₂ concentration is determined using ε₂₄₀ of 39.4 M⁻¹ cm⁻¹ (25) and that of DMPO using ε₂₂₇ of 8×10³ M⁻¹ cm⁻¹ in water. Kalyanaraman et al. (1982) Photochem. Photobiol. 36(1):5-12. Stock solution of MNP (10 mM in dimers) is prepared in 0.1 M phosphate buffers (pH 7.0 and 8.0) containing DTPA (0.1 or 0.2 mM) by stirring overnight in a vessel protected from light. This procedure generates a significant amount of MNP monomers capable of trapping radicals. Stock solutions of other reagents are prepared in glass-distilled water.

### EXAMPLE 2

### Peroxidase and Antioxidant Metabolism of β₂-Agonists

Spectrophotometric Measurements. Spectra are measured using an Agilent diode array spectrophotometer model 8453 (Agilent Technologies, Inc., Santa Clara, Calif.). Oxidation of β₂-agonists is studied by measuring absorption spectra at designated time points following reaction starts. Samples are prepared in 50 mM acetate buffer (pH 5.0), 50 and 100 mM phosphate buffers (pH 7.0 and 8.0) and 100 mM Tris/HCl (pH 9.19). All buffers contain DTPA (100 and 200 μM) and measurements are performed at an ambient temperature of 20° C. The reaction is started by the addition of a small aliquot of H₂O₂ (2, 5 or 10 μL), or glucose oxidase (1 μL), if glucose/glucose oxidase was used to generate H₂O₂, to a sample containing a studied compound, peroxidase and, if required, an inhibiting co-factor. Time course measurements are carried out following changes in absorbance at 315 nm in 15 second intervals versus absorbance at 800 nm, where none of the compounds absorb. The 315 nm wavelength is chosen because β₂-agonists' oxidation products absorb intensely near 315 nm, and because it is close to the absorption maximum of tyrosine dimers.

In certain experiments, oxidation of β₂-agonists by peroxidases is carried out using H₂O₂ generated by the reaction of glucose (1 mM) with glucose oxidase (0.2 μg/mL). The rate of H₂O₂ generation in these systems was estimated based on the rate of oxidation of ABTS (1 mM) to the green ABTS radical cation (ABTS^{•+}) by HRP, at increasing concentrations of the enzyme. Concentrations of glucose and glucose oxidase are the same as those used in experiments with β₂-agonists. The plot of the rate of ABTS^{•+} oxidation at 420 nm (determined from the linear portion of kinetic runs) versus [HRP] is a curve, which plateaus above a certain threshold value [HRP]. The mean value of the rate from the plateau region (dA₄₂₀/dt=ε₄₂₀×d[ABTS^{•+}]/dt) is taken as the rate at which all H₂O₂ produced glucose/glucose was immediately used up by the enzyme to oxidize ABTS. Calculations are performed using ε₃₁₅ (ABTS^{•+} of 3.6×10⁴ M⁻¹ cm⁻¹) (Childs et al. (1975) Biochem. J. 145:93-103), assuming that stoichiometry for the reaction is 1 mole of H₂O₂ to 2 moles of ABTS. The rate of H₂O₂ generation determined in this way is 3.33 μM/min, based on two separate determinations.

Because the commercially available fenoterol exists in the form of hydrobromide, and because bromide anion (Br⁻) is converted by peroxidases to brominating hypobromous acid (HOBr), there was the possibility that Br⁻ might interfere with enzymatic oxidation of the studied drugs. However, experiments performed in the presence of taurine and L-methionine (traps for HOBr), as well as experiments with additional doses of bromide (as NaBr) added to the sample, do not reveal any meaningful changes in the oxidation kinetics of fenoterol. Therefore, it is concluded that Br⁻ that is naturally present in the sample does not influence significantly the metabolism of fenoterol. To evaluate the role of oxygen in oxidative processes, spectrophotometric experiments were performed after bubbling N₂ gas through the sample (1 mL volume) for 5 minutes before start of the reaction (H₂O₂ addition) and then between readouts, which are collected every 1 minute.

EPR Measurements. EPR spectra are recorded using a Brüker EMX EPR spectrometer (Brüker Biospin Co., Billerica, Mass.), operating in X band and equipped with a high sensitivity resonator ER 4119HS.

Formation of free radicals from β₂-agonists is studied in samples prepared in 100 mM phosphate buffer (pH 7.0 and 8.0)/DTPA (0.2 mM) (total volume 250 μL) containing MNP, MPO (or LPO) and the agonists. The reaction is initiated by the addition of H₂O₂ as the last component. In experiments in which H₂O₂ was generated using glucose (1 mM) and glucose oxidase (3.9 μg/mL), glucose oxidase is added as a last component. The sample is transferred to a flat aqueous EPR cell and recording is started 1 minute after initiation of the reaction. Typically, spectra of MNP adducts are recorded using microwave power 20 mW, modulation amplitude 0.1 mT, receiver gain 2×10⁵, conversion time 40.96 ms, time constant 81.92 ms, and scan rate of 10 mT/41.92 s. EPR spectra constitute an average of 5 scans and represent results of typical experiments. Unless stated otherwise, direct EPR measurements (spin traps omitted) of free radicals derived from β₂-agonists are performed using conditions similar to those for the detection of MNP adducts. EPR spectra are simulated using WINSIM software developed at NIEHS/NIH (RTP, NC).

The effect of methimazole and dapsone on the formation of free radicals from drugs is studied in phosphate buffers (pH 7.0 and 8.0) containing DTPA (0.2 mM) and MNP (10 mM in dimers). Oxidation is carried out by MPO (0.43 units/mL)/H₂O₂ (37 μM) and LPO (0.39 μM)/glucose (1 mM)/glucose oxidase (0.8 μg/mL). In experiments involving methimazole, the concentrations of fenoterol and terbutaline are 0.47 mM, while in those involving dapsone, concentrations are 0.047 mM.

The effects of AscH⁻ and GSH on the metabolism of drugs are investigated using samples in 100 mM phosphate buffer (pH 7.0)/DTPA (0.1 mM) (total volume 250 μL) containing salbutamol or fenoterol and MPO, and the reaction is initiated by addition of H₂O₂ as the last component. When the effect of GSH is studied, the spin trap DMPO (18 mM) is also present. The sample is transferred to a flat aqueous EPR cell and recording is started one minute after initiation of the reaction. The spectra of DMPO adducts are recorded using the same parameters as above but the sweeping rate is 8 mT/41.92 s. The EPR spectra of ascorbate radicals are obtained using microwave power 5 mW, modulation amplitude 0.05 mT, scan rate 4 mT/41.92 s. EPR spectra shown are an average of 5 scans and represent results of a typical experiment.

### EXAMPLE 2a

### Effects of Ascorbate and Glutathione on Oxidation of Salbutamol and Fenoterol

Given that the respiratory tract lining fluid contains antioxidants such as ascorbate (AscH⁻) and glutathione (GSH) (Cross et al. (1994) Environ. Health Perspect. 102(suppl. 10):185-191), it is expected that they could affect oxidation of β₂-agonists. The concentration of AscH⁻ in airway fluid was estimated to be near 100 μM (Id.), so we use concentrations within this physiological range. FIG. 1 shows changes in A₃₁₅ versus time for 1 mM salbutamol (section A) and 200 μM fenoterol (section B) reacting with 200 mU/mL MPO and 50 μM H₂O₂ in the presence of 0, 10, 20, 40 and 100 μM AscH⁻ (FIG. 1, traces a-e, respectively) with measurements taken every 30 seconds. AscH⁻ affects oxidation of salbutamol and fenoterol similarly, causing delay in the net oxidation of both drugs. A₃₁₅ increases after a lag period, the duration of which depends on AscH⁻ concentration. Net oxidation of the drugs occurs only when AscH⁻ is consumed. At 100 μM AscH⁻, oxidation of both salbutamol and fenoterol samples is completely inhibited. This is understandable given that the concentration of H₂O₂ was only 50 μM and the peroxide was used to oxidize both the drugs and AscH⁻. The observation that oxidation of β₂-agonists resumes after an initial lag period suggests that the delay is due to interaction of AscH⁻ with the phenoxyl radicals that derive from the drugs. The proposed mechanisms of inhibition are reactions A and B, shown below, which reveal that recovery of the drug occurs at the expense of AscH⁻, which is oxidized to the ascorbate radical (A^{•-}).

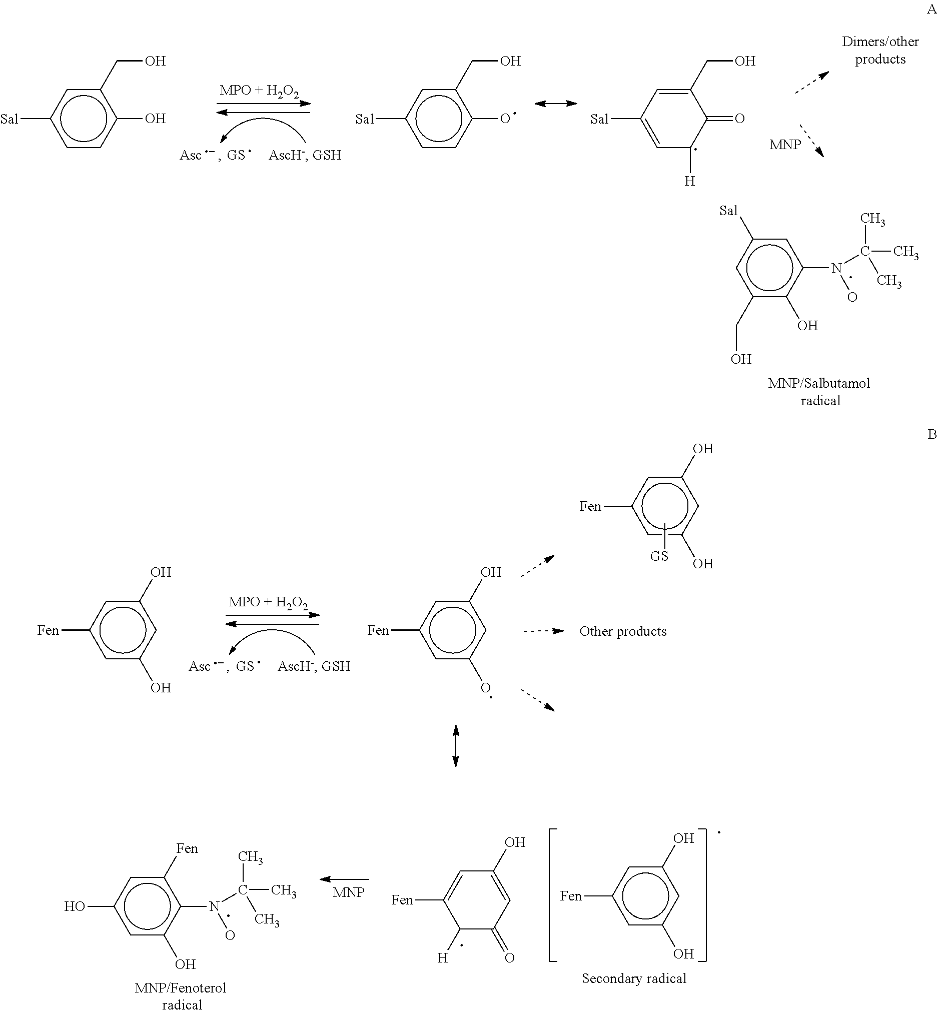

Path A shows the reaction of salbutamol, a mono-phenolic β₂-agonist. Path B shows the reaction of fenoterol, a poly-phenolic β₂-agonist.

Formation of Asc^{•-} during oxidation of β₂-agonists by MPO/H₂O₂ in the presence of 100 μM AscH⁻ is investigated by EPR. FIG. 2 displays the EPR spectra of a number of reactions. Spectrum A is the EPR of 100 μM ascorbate without additives. Sprectrun B is the EPR spectrum of 100 μM ascorbate in the presence of MPO (0.01 U/250 μL) and H₂O₂ (39 μM). Spectra C and D are the EPR spectra of 100 μM ascorbate in the presence of MPO (0.01 U/250 μ), H₂O₂ (39 μM), and 40 μM and 100 μM of salbutamol, respectily. Spectrum E is the EPR spectrum of 100 μM ascorbate in the presence of MPO (0.01 U/250 μL), H₂O₂ (39 μM) and 20 μM of fenoterol. All reactions occur in pH 7.0 buffer (50 mM) containing DTPA (0.2 mM).

Ascorbate radicals are relatively stable and produce a distinct EPR spectrum, a doublet with hyperfine splitting constant of 0.18 mT. Oxidation of AscH⁻ by MPO/H₂O₂ alone is relatively inefficient, as evidenced by the weak EPR signal of Asc^{•-} generated by the system (FIG. 2, spectrum B). In contrast, EPR spectra generated by oxidation of AscH⁻ in the presence of 40 μM and 100 μM salbutamol are approximately 76% and 117% more intense than the spectra of oxidation of AscH⁻ by MPO/H₂O₂ alone (FIG. 2, spectrum C and D, respectively), and oxidation of AscH⁻ in the presence of 20 μM fenoterol is 270% more intense than the spectra of oxidation of AscH⁻ by MPO/H₂O₂ alone (FIG. 2, spectrum E), indicating that both agonists stimulate oxidation of AscH⁻, with fenoterol being substantially more effective. Spectrum A in FIG. 2 shows that, when MPO and H₂O₂ are absent, the level of Asc^{•-} is below the detection limit.

Table 1, below, shows the efficacy of oxidation of 1 mM fenoterol and 1 mM salbutamol by 200 mU/mL MPO and 50 μM H₂O₂ in 50 mM phosphate buffer (pH 7.0) containing 0.1 mM DTPA in the presence of modulating co-factors. The extent of oxidation is expressed as ΔA₃₁₅±SE versus control (in %) during 22 minutes of reaction at 20° C. Values are the mean of at least duplicate determinations.

TABLE 1

| ΔA₃₁₅ (%) | Fenoterol | Salbutamol |
|---|---|---|
| Control | 100 | 100 |
| Catalase (235 U/mL) | 0.0 | 0.0 |
| NaCN (1 mM) | 0.24 ± 0.32 | 12.5 ± 9.1 |
| NaN₃ (1 mM) | 17.9 ± 4.2 | 6.6 ± 1.3 |
| GSH (0.1 mM)^{a} | 43.3 ± 14.5 | 62.0 ± 17.3 |
| BSA (0.5 mg/mL) | 110 ± 2.0 | 99.0 ± 2.0 |

^{a}In experiments with GSH, concentrations of salbutamol and fenoterol were 100 μM each and the reaction was continued for 30 minutes.

Spectrophotometric measurements show that GSH inhibits oxidation of β₂-agonists by MPO and H₂O₂. Because GSH is a poor peroxidase substrate, the most likely mechanism of the inhibition is interaction of the primary metabolites, phenoxyl radicals, with the thiol as depicted in reactions A and B, presented earlier. Reactions are accompanied by the formation of GS^{•} radicals as described for tyrosine and other phenolics. See, e.g., Sturgeon et al. (1998) J. Biol. Chem. 273(46):30116-30121. To verify that this mechanism operates also for β₂-agonists, EPR experiments are combined with spin trapping to detect GS^{•} radicals. When GSH is exposed to MPO/H₂O₂ in the presence of the spin trap DMPO and salbutamol, EPR spectra of DMPO/^{•}SG adduct are detected. The hfsc's a_{N}=1.51 mT, a^{β}_{H}=1.61 mT are in agreement with those determined in earlier reports for the same DMPO adduct. See, e.g., Sturgeon et al. (1998) J. Biol. Chem. 273(46):30116-30121; Harman et al. (1986) J. Biol. Chem. 261(4):1642-1648; Reszka et al. (1999) Free Radic. Biol. Med. 26(5-6):669-678

FIG. 3, section A, displays spectra recorded when 0, 80, 400, and 800 μM salbutamol is reacted with 100 μM DTPA, 18 mM DMPO, 4 mM GSH, and 0.01 U/250 mL MPO in a 50 mM phosphate buffer (pH 7.0) (spectra a-d, respectively). Spectrum e is the same reaction as spectrum d with the exception that no GSH is used. The varying spectra show that salbutamol enhances oxidation of GSH to GS^{•} in a concentration-dependent manner, as evidenced by more intense EPR spectra of DMPO/^{•}SG adducts. No radicals are detected when salbutamol alone (GSH omitted) is incubated with MPO and H₂O₂ (FIG. 3, section A, spectrum e), perhaps due to a low efficacy of the addition of the drug-derived phenoxyl radicals to DMPO or a poor stability of the resulting adduct.

When 2 μM and 20 μM fenoterol is reacted with 100 μM DTPA, 18 mM DMPO, 4 mM GSH, and 0.01 U/250 mL MPO in a 50 mM phosphate buffer (pH 7.0), EPR spectra shown in FIG. 3, section B (spectra a and b, respectively) are detected. Although the general pattern is similar to that observed with salbutamol, fenoterol appears to be markedly more efficient in stimulating oxidation of GSH. The EPR spectrum generated in the presence of 2 μM fenoterol (FIG. 3, section B, spectrum a) is approximately a two-fold more intense than that observed in the presence of 80 μM salbutamol (FIG. 3, section A, spectrum b). This confirms the higher reactivity of a metabolite(s) derived from fenoterol. When 400 μM fenoterol is used but GSH is omitted, no radicals from fenoterol are detected by spin trapping with DMPO (FIG. 3, section B, spectrum c). The higher stimulatory action of fenoterol, when compared to that of salbutamol, implies that the compound's resorcinol moiety may play a dominating role in the interaction with GSH. The proposed cycle of redox reactions involving fenoterol, AscH⁻ and GSH is depicted in reaction B, presented earlier.

FIG. 4 shows the effect of GSH and BSA on oxidation of fenoterol and terbetuline. Spectrum a shows the absorbance spectrum of intact fenoterol; spectrum b shows the absorbance spectrum of oxidized fenoterol. Spectrum c shows the absorbance spectrum of fenoterol oxidized in the presence of 100 μM GSH.

Oxidation of fenoterol in the presence of GSH generates a new species with absorption maximum at 395 nm (FIG. 4, spectrum c), which shifts to 375 nm (FIG. 4, spectrum d) after further incubation. The new species is tentatively ascribed to a fenoterol-SG conjugate. Because oxidation of salbutamol in the presence of GSH does not produce this spectral feature, the fenoterol resorcine moiety might be involved. We performed spectrophotometric analysis of terbutaline oxidized in the presence of GSH, since it too possesses resorcine moiety. As with fenoterol, terbutaline exhibits a spectrum with an intense maximum at 365 nm, confirming that the resorcine portion of these molecules participates in the reaction with GSH. Formation of conjugates with thiols has been described for para- and ortho-quinones. See, e.g., Takahashi et al. (1987) Arch. Biochem. Biophys. 252(1):41-48; Rao et al. (1988) J. Biol. Chem. 263(34):17981-17986. Because oxidation of meta-hydroxybenzenes cannot lead to meta-quinones, formation of a fenoterol (or terbutaline) conjugate with GSH has to involve another intermediate, capable of addition the thiol, possibly a tri-hydroxybenzene, formed in situ in the system. Formation of such an intermediate is suggested by analysis of EPR spectra of radicals formed during oxidation of fenoterol and metaproterenol.

The role of BSA in the metabolism of β₂-agonists is also examined. As determined by measuring A₃₁₅, BSA (0.5 mg/mL) minimally affected oxidation of salbutamol and fenoterol by MPO/H₂O₂, essentially increasing A₃₁₅ (Table 1). The absorption spectrum of fenoterol oxidized in the presence of 0.5 mg/mL BSA (FIG. 4, spectrum e) closely resembles the spectrum of fenoterol oxidized in the presence of 100 μM GSH (FIG. 4, spectrum d), after a prolonged incubation. This suggests that oxidized fenoterol may form a conjugate with BSA, presumably through addition to the protein thiol group. Spectrum f shows the absorbance spectrum of terbetuline oxidized in the presence of 100 μM GSH.

### EXAMPLE 2b

### Effect of Inhibitors on Oxidation of β₂-Agonists by LPO/H₂O₂

Table 2, below, shows the effect of inhibitors on the oxidation of fenoterol and salbutamol by LPO/H₂O₂. H₂O₂ is generated by 1 mM glucose and 0.2 μg/mL glucose oxidase. Oxidation of 50 μM fenoterol and 100 μM salbutamol is carried out in 0.1 M potassium phosphate buffer (pH 7.0) containing 0.1 mM DTPA in the presence of LPO (158 nM LPO for fenoterol and 216 nM for salbutamol). The extent of inhibition is determined by measuring ΔA₃₁₅ during 30 minutes of reaction and is expressed as % of control (mean±SE from at least two determinations).

TABLE 2

| | Amount of metabolite formed | |
|---|---|---|
| | ΔA₃₁₅ (%) | |
| | Fenoterol | Salbutamol |
| Control | 100 | 100 |
| NaN₃ (1 mM) | 45.5 ± 3.9 | 17.4 ± 7.1 |
| NaCN (1 mM) | 95.2 ± 5.3 | 80.0 ± 3.2 |
| NaSCN (0.1 mM) | 22.8 ± 3.8 | 5.5 ± 7.3 |
| GSH (0.1 mM) | 38.1 ± 3.2 | 42.7 ± 3.4 |
| Methionine (0.1 mM) | 103.7 ± 2.0 | 105.5 ± 16.2 |
| Methionine (2 mM) | 95.0 ± 3.5 | — |
| Methimazole (20 μM) | 30.0 ± 2.9 | 16.7 ± 4.6 |

Oxidation of salbutamol and fenoterol by LPO/glucose/glucose oxidase is strongly inhibited by azide (from NaN₃), but weakly inhibited by cyanide (from NaCN). Both thiocyanate (from NaSCN), the natural substrate for LPO, and GSH markedly inhibit oxidation of fenoterol and salbutamol. L-methionine, in which the thiol group is methylated, is inactive, emphasizing the importance of the free —SH group for effective antioxidant action. Because LPO-catalyzed oxidation could be a mechanism that inactivates β₂-agonists in airways, we sought to determine whether pharmacological inhibitors of peroxidase could affect oxidation of these drugs.

Methimazole, an antithyroid drug, is known to inhibit thyroid peroxidase and LPO.

Methimazole markedly inhibits oxidation of both salbutamol and fenoterol by the LPO system (Table 2), primarily due to inactivation of the enzyme, as shown by the marked decrease in the intensity of the LPO Soret band.

### EXAMPLE 2c

### Effect of Peroxidase Inhibitors on Free Radical Formation—EPR Study

Table 3, below, summarizes the effects of dapsone and methimazole on EPR spectra of MNP adducts with radicals from fenoterol and terbutaline generated by MPO/H₂O₂ and LPO/H₂O₂ systems at a pH of 8.0. Concentrations of MNP spin adducts were calculated as second integrals of the low-field component of the respective spectra and are expressed as % of control samples (no additives). Results equal the means±SE of at least two measurements.

TABLE 3

| | +MPO + H₂O₂ | +LPO + H₂O₂ |
|---|---|---|
| | Fenoterol | |
| −No additives | 100^{a} | 100^{b} |
| +Dapsone (1 mM) | NA | 13.9 ± 0.6^{a} |
| +Methimazole (0.86 mM) | 22.4 ± 0.6^{a} | 26.9 ± 8.2^{b} |
| | Terbutaline | |
| −No additives | 00^{a} | 100^{b} |
| +Dapsone (1 mM) | NA | 54.6 ± 20.0^{a} |
| +Methimazole (0.86 mM) | 33.5 ± 3.5^{a} | 33.9^{b} |

^{a}The oxidant was the reagent H₂O₂ (37 μM).
^{b}H₂O₂ was generated using glucose (1 mM) and glucose oxidase (0.8 μg/mL).

In experiments with methimazole, concentrations of fenoterol and terbutaline were 0.47 mM. In experiments with dapsone, such concentrations were 0.047 mM. The activity of MPO was 500 mU/mL and the concentration of LPO was 72 mM. Methimazole (0.86 mM) and dapsone (1 mM) markedly inhibit the formation of radicals from fenoterol and terbutaline oxidized by LPO/glucose/glucose oxidase (or H₂O₂ reagent). When the MPO system was used, only methimazole was inhibitory; dapsone has only a minimal effect on MPO activity. This is consistent with the known inhibitory action of both methimazole and dapsone on LPO activity and only methimazole on MPO activity. Thomas et al. (1994) J. Dent. Res. 73(2):544-55. Results obtained at pH 7.0 qualitatively agree with those at pH 8.0. These results suggest that methimazole and dapsone may have the potential to prevent the peroxidative metabolism of β₂-agonists and maintain β₂-agonist bronchodilator activity during prolonged or severe asthma exacerbation.

### EXAMPLE 3

### Nitration of Salbutamol

Exhaled breath condensate (“EBC”) samples. EBC samples are collected during 10 min of quiet breathing through a single-use disposable RTube collector (Respiratory Research, Inc., Charlottesville, Va., USA) while subjects are wearing a nose clip. The aluminum sleeve of the device is cooled to an initial temperature of −20° C. prior to collection. After collection, a plunger is used to pool the condensed material within the tube into a single sample (about 1.0 ml). Samples are stored in the reaction tube at a temperature of −80° C. until thawed and processed for analysis.

Spectrophotometric Measurements. Spectra are measured using an Agilent diode array spectrophotometer model 8453 (Agilent Technologies, Inc., Santa Clara, Calif.). Nitration of salbutamol is studied by measuring absorption spectra at designated time points following the start of the reaction. Samples are prepared in 100 mM phosphate buffers (pH 7.0). All buffers contained DTPA (100 μM) or are pretreated with Chelex-100 before use, and measurements are performed at ambient temperature of 20° C. Typically, the reaction is started by the addition of a small aliquot of H₂O₂ or the desired peroxidase. Time course measurements are carried out following changes in absorbance at 410 nm in 30 second intervals versus absorbance at 800 nm, where none of the compounds absorb.

To determine molar absorptivity of the mixture of salbutamol-derived metabolites, the absorbance at 410 nm is measured following multiple additions of peroxidases, H₂O₂, and NO₂⁻until A₄₁₀ stabilized, indicating that all salbutamol was consumed. These experiments are conducted at various initial concentrations of the drug. Based on these measurements, ε₄₁₀ for the mixture of metabolites was estimated to be 4550±227 M⁻¹ cm⁻¹ (N=14). The effect of pH on ionization of the compound's phenolic moiety was determined by measuring absorption spectra of samples prepared by adding equal volumes of nitrated salbutamol (25 μL) to buffers (0.1 M, 475 μL) of pH ranging from 4 to 9.

Mass Spectrometry. The experiment is performed on a Thermo Fisher Scientific LTQ-FT, a hybrid instrument consisting of a linear ion trap and a Fourier transform ion cyclotron resonance mass spectrometer. Liquid chromatography separation of the sample components utilizes a Waters XBridge™ C18 3.5 μm 2.1×100 mm column, Finnigan Surveyor MS pump, and Finnigan Micro AS autosampler. A 200 μL/min gradient elution of water and acetonitrile, both containing 0.1% formic acid, occurs as follows: 5% to 32% acetonitrile in first eight minutes followed by a rapid rise to 95% acetonitrile within the next minute, held at 95% for an additional seven minutes. The entire elutant is introduced into the LTQ-FT using the standard electrospray ionization source for the instrument with a spray voltage of 5 kV and a capillary temperature of 275° C. Autogain control (hereinafter “AGC”) is used and set at 500,000 with a maximum injection time of 1250 ms for FT-ICR full scans. Collision-induced dissociation, MS/MS, was executed in the linear trap with an AGC setting of 10000 and a maximum injection time of 500 ms. FT-ICR full scans are acquired in the positive ion mode at 100,000 resolving power at m/z 400. Mass accuracy errors for salbutamol and its nitrated analogs are below 250 ppb. The positive ion MS/MS experiments are performed simultaneously in the linear trap portion of the instrument using helium as a collision gas, isolation widths of 2 amu, normalized collision energies of 35 to 40, a q value of 0.250 and an excitation time of 30 ms.

Samples for mass spectrometry experiments are prepared using ˜50 μM salbutamol, 5.9 mM H₂O₂, 5.8 mM NaNO₂ and 0.25 μl tIVI LPO (or 0.19 U/mL MPO) and progress of the reaction was monitored measuring A₄₁₀, and continued until A₄₁₀ ceased to increase. Before the MS analysis, proteins are removed using Centricon® centrifugal filter device with 10 kD cut-off filter (Millipore).

β₂-AR receptor binding and cAMP generation. Receptor affinity is assessed by measuring the ability of native and nitrated salbutamol to displace binding of the β₂AR antagonist ¹²⁵I-CYP as previously described. McGraw et al. (1997) J. Biol. Chem. 272(11):7338-7344. Binding assays are carried out with crude membrane preparations from airway smooth muscle cells that transgenically overexpress the human β₂-AR.

The ability of native and nitrated salbutamol to stimulate cAMP production in airway smooth muscle cells is measured by a fluorescent detection assay using a commercially available kit (Mediomics, St. Louis, Mo.). For these studies, cells are grown to near-confluence in 96-well plates. After washing with PBS, cells were treated for 15 minutes with either PBS vehicle or the indicated concentration of isoproterenol. The reaction is then halted using the supplied stop buffer, after which the resultant sample is processed for fluorescent detection per the manufacturer's instructions. The cAMP content for each sample is determined by extrapolating values to a standard curve prepared with known concentrations of cAMP.

### EXAMPLE 3a

### Salbutamol Transformation In Vivo

Mass spectrometry analysis is performed on exhaled breath condensate samples from asthmatic patients undergoing salbutamol therapy. FIG. 5, shows HPLC elution profile with a peak at 6.80 min (trace A), which coincides with the HPLC peak (6.75 min) from salbutamol-derived nitrophenol of m/z 255.13 (trace B). The MS/MS spectrum of this molecular ion is shown in FIG. 10, trace C. The product ions of m/z of 237.17, 199.00, 181.00, and 130.25 are detected and this fragmentation pattern is similar to that from the ion of the same retention time from salbutamol nitrated in vitro (FIG. 5, trace D). suggesting that the presence of oxidatively modified salbutamol in breath condensates of asthmatic patients. Together, the data (the same retention times, the same parent ions of m/z 255.13, and the same fragments observed in MS/MS from both samples) strongly indicate that, in the airway environment of asthmatic patients, salbutamol undergoes oxidation and nitration.

### EXAMPLE 3b

### Effect of Salbutamol Transformation on β₂-AR binding and cAMP Generation

The ability of β₂-agonists such as salbutamol to stimulate receptor signaling is dependent upon their ability to bind to the receptor and induce conformational changes in β₂-AR structure that facilitate activation of its associated G-proteins. Addition of a nitrite moiety to salbutamol, or formation of salbutamol dimers, could potentially impair agonist-mediated signal transduction by either altering salbutamol's ability to bind to the β₂-AR, or alternatively, the nitrated agonist might bind the receptor but fail to induce the conformational changes in receptor structure necessary for signal transduction. We therefore perform competition binding assays using airway smooth muscle cells derived from transgenic mice that overexpress the human β₂-AR to determine whether the affinity of nitrosalbutamol for the β₂-AR was different from that of the native drug. As shown in FIG. 6, section A, salbutamol transformed by LPO/H₂O₂/NO₂⁻ displaces binding of the nonselective β₂AR antagonist ¹²⁵I-CYP binding to airway smooth muscle cell membranes by nearly two orders of magnitude less than that of native salbutamol, indicating that nitration of the parent compound markedly reduced its capacity to bind the β₂-AR.

To establish whether the reduction in receptor affinity is associated with decreased signal transduction, we compare the ability of salbutamol and its nitrosalbutamol to stimulate cAMP synthesis in murine airway smooth muscle cells. Consistent with the reduction in binding affinity, we find that the ability of the nitrosalbutamol to stimulate cAMP in these cells was approximately three orders of magnitude less than that of the parent compound (FIG. 6, section B), with EC₅₀ 2.98±0.09×10⁻⁵ M versus 2.48±0.05×10⁻⁸ M, respectively.

### EXAMPLE 3c

### Effect of Ascorbate and Thiocyanate on Nitration of Salbutamol

Ascorbate (AscH⁻) is an effective natural antioxidant protecting airways from oxidative injury. Cross et al., (1994) Environ. Health Perspect. 102 (suppl. 10):185-191. Its concentration in the respiratory tract lining fluid is estimated to be near 100 μM (Id.). We examine the effect of AscH⁻ on nitration of 100 μM salbutamol by 67 nM LPO, 4 mM H₂O₂, and 3.5 mM of NaNO₂ by measuring absorption at 410 nm. FIG. 7 shows changes in A₄₁₀ versus time in the presence of 0, 55, 88, and 109 μM AscH⁻ (FIG. 7, traces a-d, respectively). Ascorbate causes a delay in the appearance of the peak at 410 nm, suggesting that nitration of the β₂-agonist is also delayed. The duration of the lag period depends on AscH⁻ concentration. Only when AscH⁻ is consumed is the nitration of salbutamol observed, suggesting that the delay is not due to inhibition or inactivation of the enzyme, but rather due to the interaction of AscH⁻ with salbutamol's phenoxyl radicals or NO₂^{•} radicals. See, e.g., Reszka et al. (1999) Free Radic. Biol. & Med. 26:669-678. Thus, AscH⁻ at near physiological concentrations only transiently inhibits nitration of the drug. However, supplementation of the system with ascorbate at higher than physiological concentrations, e.g., ˜1 mM, completely prevents nitration of the drug.

Thiocyanate (SCN⁻) is a natural constituent of airway fluids. Its concentration ranges from 20 μM to 120 μM and is higher in smokers. Tenovuo et al (1976) J. Dent. Res. 55(4):661-663. Thiocyanate is believed to be a natural substrate of LPO, although it is also oxidized by MPO and EPO systems. SCN⁻ inhibits nitration of salbutamol, as shown in FIG. 8, which shows A₄₁₀ versus time traces recorded in the presence of 0, 10, 25, and 50 μM NaSCN. Under conditions used and at [NaSCN] <50 μM, SCN⁻ acts similarly to AscH⁻, inhibiting nitration only transiently (FIG. 8, traces b-c). However, 50 μM SCN completely prevented nitration of salbutamol (FIG. 8, trace d).

### EXAMPLE 3d

### Effect of Methimazole and Dapsone on Nitration of Salbutamol

As noted earlier, methimazole is an antithyroid drug, an effective inhibitor of LPO activity. 100 μM of salbutamol is reacted with 73 nM LPO, 3.5 mM NaNO₂, and 4 mM H₂O₂ at pH buffer 7.0 in the presence of 0, 27, 55, and 109 μM methimazole (shown in FIG. 9, section A, traces a-d, respectively). Traces c and d also indicate by arrow when additional LPO is added to the system. Methimazole markedly inhibits oxidation of salbutamol by LPO/H₂O₂. Methimazole inhibits nitration of salbutamol by LPO/H₂O₂/NO₂⁻ in a concentration-dependent manner, as FIG. 9 shows. In contrast to effects exerted by AscH⁻ or SCN⁻, methimazole-dependent inhibition is permanent and is probably due to inactivation of the enzyme, since a second dose of LPO reactivates the process (FIG. 9, section A, trace c). Methimazole at a concentration close to 100 μM completely prevents nitration of the drug (FIG. 9, section A, trace d).

We also examine the effect of methimazole on nitration catalyzed by MPO and EPO. 50 μM of salbutamol is reacted with 0.2 U/mL MPO, 5 mM NaNO₂, and 2.1 mM H₂O₂ at pH buffer 7.0 in the presence of 0, 25, 50, and 100 μM methimazole (shown in FIG. 9, section B, traces a-d, respectively). In contrast to inhibition of nitration by LPO system, which was irreversible, inhibitory action of methimazole on MPO/H₂O₂/NO₂⁻ is transitory. The A₄₁₀ versus time traces recorded at various concentrations of methimazole are shown in FIG. 9, section B. The S-shape of these traces indicates that methimazole affects the process only at its initial stage, presumably because it is a good MPO substrate and competes with other reactants for the active site of the enzyme. A similar effect of methimazole was observed when MPO was replaced by EPO.

Dapsone, an anti-inflammatory and anti-leprotic agent, is a potent inhibitor of LPO and MPO. See, e.g., Kettle et al. (1991) Biochem. Pharmacol. 41(10):1485-1492; Bozeman et al. (1992) Biochem. Pharmacol. 44(2):553-563. 97 μM of salbutamol is reacted with 65 nM LPO, 3.4 mM NaNO₂, and 4 mM H₂O₂ at pH buffer 7.0 in the presence of 0, 2.5, 5, 10, 50, and 100 μM dapsone (shown in FIG. 10, traces a-f, respectively). The arrows in traces d and e of FIG. 10 indicate where additional LPO is added to the system. As FIG. 10 shows, dapsone is an effective inhibitor of nitration of salbutamol by LPO/H₂O₂/NO₂⁻. Dapsone at 50 μM inhibited nitration by approximately 86%. Given the high concentrations of NaNO₂ and H₂O₂ used in this system, dapsone appears to be a potent blocker of nitration. As with methimazole, reactions resume after addition of new doses of the enzyme, indicating that inhibition by dapsone is due to inactivation of LPO (FIG. 10, traces d and e).

Table 4, below, summarizes inhibition of salbutamol nitration by peroxidase/H₂O₂/NO₂⁻ systems at various dapsone concentrations, as indicated by the relative production of nitrophenols.

TABLE 4

| | |
|---|---|
| Dapsone, μM | Nitrophenols, (%) |

LPO

| | |
|---|---|
| 0 | 100 ± 3.0 |
| 2.5 | 72.8 ± 2.0 |
| 5.0 | 58.4 ± 1.4 |
| 10.0 | 45.7 ± 2.0 |
| 50.0 | 14.0 ± 2.7 |

MPO

| | |
|---|---|
| 0 | 100 |
| 100 | 66.3 ± 0.6 |
| 250 | 56.0 ± 1.3 |
| 500 | 44.2 ± 3.0 |

EPO

| | |
|---|---|
| 0 | 100 ± 3 |
| 140 | 57 |
| 300 | 34 ± 2 |

The extent of nitration is expressed as a change in ΔA₄₁₀ versus control (dapsone omitted) taken as 100% after 10, 30, and 60 minute reactions for LPO, MPO, and EPO nitrating systems, respectively. Results equal the means±SE of at least two measurements. In the LPO system, 100 μM of salbutamol, 3.4 mM of NaNO₂, 4 mM of H₂O₂, and 65 nM of LPO is used. In the MPO system, 50 μM of salbutamol, 5.0 mM of NaNO₂, 5 mM of H₂O₂, and 0.2 U/mL of MPO is used. In the EPO system, 50 μM of salbutamol, 5 mM of NaNO₂, 2 mM of H₂O₂, and 0.075 U/mL of EPO is used. All reactions were carried out in pH 7.0 phosphate buffer containing DMSO (5% v/v). The term “nitrophenols” includes nitrosalbutamol (N-Sal) and salbutamol-derived nitrophenol (N—ArOH).

Similar experiments are carried out with MPO and EPO nitrating systems. Under comparable conditions, dapsone inhibits nitration by EPO/H₂O₂/NO₂⁻ to a higher degree than MPO/H₂O₂/NO₂⁻. EPO appears to be more sensitive to dapsone than is MPO.

Both methimazole and dapsone efficiently and permanently inhibit nitration catalyzed by LPO system, presumably by poisoning the enzyme, since additional doses of LPO reactivate the process. In contrast, the effects in an LPO system of methimazole and dapsone on the reactions catalyzed by MPO and EPO are less pronounced, even at high concentrations of the inhibitors.

It is possible to minimize the metabolism of β₂-agonists by administering peroxidase inhibitors and antioxidants, thus preserving the activity and effectiveness of the β₂-agonists.

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating a reactive airway disease in a subject comprising administering at least one peroxidase inhibitor in association with administration of at least one β₂-agonist.

2. The method of claim 1, wherein the reactive airway disease is selected from the group consisting of asthma, emphysema, and bronchitis.

3. The method of claim 1, wherein the β₂-agonist is selected from the group comprising salbutamol, fenoterol, terbutaline, isoproterenol, salmeterol, formoterol, arformoterol, and indacaterol.

4. The method of claim 1, wherein the peroxidase inhibitor is selected from the group consisting of methimazole, dapsone, and thiocyanate.

5. The method of claim 1, wherein administering the at least one peroxidase inhibitor comprises administering the at least one peroxidase inhibitor via inhalation.

6. The method of claim 5, wherein administering the at least one peroxidase inhibitor comprises administering the at least one peroxidase inhibitor via a metered dose inhaler or a nebulizer.

7. The method of claim 1, wherein administering at least one peroxidase inhibitor in association with administration of at least one β₂-agonist comprises administering the at least one peroxidase inhibitor concurrent with administration of the at least one β₂-agonist.

8. The method of claim 1, wherein administering at least one peroxidase inhibitor in association with administration of at least one β₂-agonist comprises administering the at least one peroxidase inhibitor separate from administration of the at least one β₂-agonist.

9. The method of claim 8, wherein administering at least one peroxidase inhibitor in association with administration of at least one β₂-agonist comprises administering the at least one peroxidase inhibitor prior to administration of the at least one β₂-agonist.

10. The method of claim 1, further comprising administering at least one antioxidant in association with administration of at least one β₂-agonist.

11. The method of claim 10, wherein the antioxidant is selected from the group consisting of ascorbate and glutathione.

12. The method of claim 10, wherein administering the at least one antioxidant comprises administering via inhalation.

13. The method of claim 12, wherein administering the at least one antioxidant comprises administering via a metered dose inhaler or a nebulizer.

14. The method of claim 10, wherein administering at least one antioxidant in association with administration of at least one β₂-agonist comprises administering the at least one antioxidant concurrent with administration of the at least one β₂-agonist.

15. The method of claim 10, wherein administering at least one antioxidant in association with administration of at least one β₂-agonist comprises administering the at least one antioxidant separate from administration of the at least one β₂-agonist.

16. The method of claim 10, wherein administering at least one antioxidant in association with administration of at least one β₂-agonist comprises administering the at least one antioxidant prior to administration of the at least one β₂-agonist.

17. A pharmaceutical composition comprising at least one β₂-agonist, at least one peroxidase inhibitor in an amount effective to inhibit metabolism of the at least one β₂-agonist, and a pharmaceutically acceptable carrier.

18. The pharmaceutical composition of claim 17, wherein the composition is aerosolized or nebulized.

19. The pharmaceutical composition of claim 17, further comprising at least one antioxidant in an amount effective to inhibit metabolism of the at least one β₂-agonist.

20. The pharmaceutical composition of claim 19, wherein the composition is aerosolized or nebulized.

21. An inhaler comprising:
at least one β₂-agonist, and
at least one peroxidase inhibitor in an amount effective to inhibit metabolism
of the at least one β₂-agonist.

22. The inhaler of claim 21, further comprising at least one antioxidant in an amount effective to inhibit metabolism of the at least one β₂-agonist.
